SEQUENCE LISTING (8 sequences)

SEQ ID NO: 1 - DNA, 49 bp, Artificial sequence
  Description of the artificial sequence Oligonucleotide phosphorylated in position 5′ derived from the gene sequence coding for thymidylate synthase
tggataaaatggcgctggcaccggtacatgcattcttccagttctatgt

SEQ ID NO: 2 - DNA, 49 bp, Artificial sequence
  Description of the artificial sequence Oligonucleotide phosphorylated in position 5′ derived from the gene sequence coding for thymidylate synthase
tggataaaatggcgctggcaccgatacatgcattcttccagttctatgt

SEQ ID NO: 3 - DNA, 18 bp, Artificial sequence
  Description of the artificial sequence Oligonucleotide derived from the gene sequence coding for thymidylate synthase
ggtgtgatcatgatggtc

SEQ ID NO: 4 - DNA, 18 bp, Artificial sequence
  Description of the artificial sequence Oligonucleotide derived from the gene sequence coding for thymidylate synthase
cctgcaagatggattccc

SEQ ID NO: 5 - DNA, 19 bp, Artificial sequence
  Description of the artificial sequence Oligonucleotide derived from the gene sequence coding for thymidylate synthase
cgcgccgcattattgtttc

SEQ ID NO: 6 - DNA, 19 bp, Artificial sequence
  Description of the artificial sequence Oligonucleotide derived from the gene sequence coding for thymidylate synthase
gtctggaccggtggcgaca

SEQ ID NO: 7 - DNA, 33 bp, Artificial sequence
  Description of the artificial sequence Oligonucleotide phosphorylated in position 5′ derived from the gene sequence coding for valyl-tRNA synthetase
ggggaattcggtgtgtgaaattgccgcagaacg

SEQ ID NO: 8 - DNA, 33 bp, Artificial sequence
  Description of the artificial sequence Oligonucleotide phosphorylated in position 5′ derived from the gene sequence coding for valyl-tRNA synthetase
ggcaagcttccagtatttcacggggagttatgc

(19) United States
(12) Patent Application Publication
(10) Pub. No.: US 2004/0014942 A1
(43) Pub. Date: Jan. 22, 2004

(54) Mutant strains capable of producing chemically diversified proteins by incorporation of non-conventional amino acids

(76) Inventors: Philippe Marliere, Etiolles (FR); Volker Doring, Paris (FR); Henning Mootz, Marburg (DE)

Correspondence Address: FINNEGAN, HENDERSON, FARABOW, GARRETT & DUNNER, LLP, 1300 I STREET, NW, WASHINGTON, DC, 20005, US

(21) Appl. No.: 10/258,795

(22) PCT Filed: Apr. 27, 2001
(86) PCT No.: PCT/FR01/01306

(30) Foreign Application Priority Data
Apr. 28, 2000 (FR) 0005547

(51) Int. Cl. C07K 1/00; C07K 14/00; C07K 17/00

(52) U.S. Cl. 530/350

(57) ABSTRACT

The invention concerns mutant prokaryotic cells, in particular E. coli, capable of producing proteins whereof the amino acid sequences comprise at least a non-conventional amino acid, methods for producing and purifying said proteins and the proteins obtained by said methods. The invention also concerns uses of said cells and proteins in different fields such as in therapy, cosmetics, diagnosis or biosynthesis or biodegradation of organic compounds.

The present invention concerns mutant prokaryotic cells, in particular E. coli, which are capable of producing proteins whereof the amino acid sequences include at least one non-conventional amino acid, methods for producing and purifying said proteins and the proteins obtained by the methods according to the invention. The invention also covers applications of said cells and proteins in different fields, such as in therapy, cosmetics, diagnosis or biosynthesis or biodegradation of organic compounds.

A growing number of proteins produced in large quantities by recombinant organisms are used as catalysts in the chemicals industry or as therapeutic agents. The search for new proteins with diversified functions is the subject of intense activity, either screening the proteins of extremophilic organisms, or creating protein variants by mutagenesis and screening. However the chemical variability of the proteins which can be produced in living organisms remains limited by the invariance of the genetic code, i.e. restricted to the combinations of a canonical set of 20 amino acids. If the descent of natural species could be gradually remodelled in the laboratory in such a manner as to adopt different genetic codes, the evolution of the proteins could be redirected and artificial sources of biodiversity could thus be established.

Experimental deviation from the genetic code is the only way that this limitation could be overcome. Another genetic code could specify a larger or smaller set of amino acids, a set replaced by non-canonical monomers or a set of canonical amino acids among which the codons are redistributed. The specification of additional amino acids in living lines would lend itself to numerous applications, the most generic being the establishment of an artificial biodiversity.

The permanent or temporary incorporation of a single additional amino acid bearing a chemical motif which could react without modifying the conventional amino acids, would suffice to establish new methods of protein functionalisation. This is precisely the subject of the present invention.

The invention concerns a method enabling cells to acquire the capacity to produce a protein whereof the amino acid sequence includes at least one non-conventional amino acid, characterised in that it comprises the following steps:

a) the transformation of said cells by the introduction of at least one false-sense mutation at the target codon of a gene coding for a protein necessary for the growth of said cells, said protein being synthesised from the gene thus mutated no longer being functional;

b) where appropriate the culture of the cells obtained in stage a) in a culture medium containing the nutrient made necessary by the loss of functionality of said protein thus mutated; and

c) the culture of the cells obtained in stage a) or b) in a culture medium containing the amino acid coded by said target codon.

In the present description the term protein is intended to refer to peptides or polypeptides equally, as well as the corresponding glycoproteins when said proteins are glycosylated.

In the present description the term non-conventional amino acid is also intended to refer to any amino acid other than the amino acids incorporated by the ribosomes during the biosynthesis of the proteins synthesised by prokaryotic or eukaryotic unicellular or multicellular organisms, as well as any amino acid incorporated in the place of the amino acid which should normally be incorporated in this place with regard to the translated nucleic sequence.

Also in the present description the term false-sense mutation is intended to refer to a mutation which transforms a codon representing an amino acid into a codon which codes for another amino acid, the latter, where appropriate, not being able to replace the original amino acid to provide a functional protein in the protein in the place of the residue of the original amino acid.

Also in the present description the term protein necessary for the growth of cells is intended to refer to a protein which, when it is synthesised by the cells in a functional manner, allows said cells to grow in given culture conditions and which, when it is synthesised by the cells in a non-functional manner, necessitates the introduction of a supplementary nutrient into said given culture medium to allow said cells to grow. Such non-functional proteins can for example be synthesised by cells following conditional mutations such as a mutation of the photosensitive type.

To illustrate this by an example, but without being limited thereto, it is possible to cite in particular the thymidylate synthase protein of E. coli which presents a catalytic site occupied by cysteine at position 146 of its amino acid sequence, and whereof the corresponding mutations of the gene (thyA) cause a nutritional requirement for thymine or thymidine, no other amino acid being able to replace the cysteine at this site.

In the present description, the term target codon is intended to refer to the codon with three nucleotide bases transformed by false-sense mutation, said target codon being the sequence of 3 bases before transformation by said false-sense mutation.

The invention also includes a method according to the invention, characterised in that the culture medium of stage c) does not contain the nutrient required by the loss of functionality of said mutated protein.

According to the invention, stage c) in the culture of said cells can comprise a series of cultures of said cells in a culture medium containing the amino acid coded by said target codon (before its transformation by said false-sense mutation) each of said cultures in the series being effected up to the obtaining of the stationary growth phase and followed by a washing of the cells obtained, the number of cultures in the series being sufficient to allow the selection of mutations increasing the suppression of said false-sense mutation of said mutated gene and the propagation of the allele corresponding to said mutated gene.

The invention further relates to a method according to the invention, characterised in that the false-sense mutation is chosen from the false-sense mutations which reverse spontaneously with only very low frequency, of the order of one organism out of at least 10¹⁵.

The false-sense mutation will preferably be chosen from the false-sense mutations which transform a target codon of a gene coding for a protein necessary for the growth of said cell into a codon which in comparison with the target codon exhibits a change of at least two bases, more preferably three bases.

Also preferred are the methods according to the invention, characterised in that the target codon codes for an amino acid which has a low steric volume and/or is amphiphilic and/or has a steric volume lower than or roughly equal to the steric volume of the amino acid coded by the false-sense mutation.

Out of the target codons, those preferred in particular are the target codons coding for cysteine and false-sense mutations chosen from the false-sense mutations which transform a target codon into a codon coding for valine or isoleucine.

The invention further relates to a method according to the invention, characterised in that stage a) in the transformation of said cells is achieved by means of a vector comprising a sequence of said gene coding for a protein necessary for the growth of said cells comprising said false-sense mutation, in particular by means of a plasmid vector.

Such vectors will be prepared according to the methods currently used by the person skilled in the art, and the resultant clones can be introduced into said cells by the usual gene recombination methods, such as for example lipofection, electroporation or thermal shock.

From another perspective, the invention concerns a method of selecting cells capable of producing a protein whereof the amino acid sequence includes at least one non-conventional amino acid characterised in that it comprises stages a), and where appropriate b) and c) of a method according to the invention, and the selection of the cells capable of growing at stage c).

In a preferred manner, the method of selecting cells according to the invention will in addition include a stage d) of culture of the cells obtained at stage c) in a culture medium containing said amino acid coded by said target codon, the concentration of said amino acid possibly being at a concentration higher than the concentration of said amino acid used in stage c), and the choice of cells sensitive to the concentration of said amino acids used in stage d).

The term cell sensitive to a chemical or biochemical compound or to a given concentration of said compound is intended to refer to a cell whose growth is partially or totally inhibited when it is cultivated in a culture medium containing said chemical or biochemical compound or said concentration of said compound.

The invention also includes a method of selecting cells according to the invention, characterised in that the aminoacyl-tRNA synthetase recognising the amino acid coded by said false-sense mutation of said selected cells is capable of charging one of its associated tRNA's with a non-conventional amino acid or an amino acid other than said amino acid coded by said false-sense mutation.

In the present description, the term associated tRNA is intended to refer to a tRNA which is recognised by the aminoacyl-tRNA synthetase recognising an amino acid and which can transfer said amino acid.

The invention further includes a method of selecting mutant cells according to the invention, characterised in that the nucleic sequence of the gene coding for said aminoacyl-tRNA synthetase includes at least one mutation in comparison with the sequence of the corresponding wild-type gene, said mutation not having been introduced by a gene recombination technique.

From another perspective, the invention concerns prokaryotic or eukaryotic cells obtained by a method according to the invention.

Of the cells which can be used for these purposes, mention may of course be made not only of bacterial cells such as E coli, but also yeast cells, as well as animal cells, in particular cultures of mammal cells, such as in particular Chinese hamster ovary (CHO) cells, and also of insect cells.

The invention also relates to isolated prokaryotic or eukaryotic cells capable of producing a protein whereof the amino acid sequence includes at least one non-conventional amino acid, characterised in that they include an aminoacyl-tRNA synthetase recognising a given amino acid capable of charging one of its associated tRNA's with a non-conventional amino acid or an amino acid other than said given amino acid, and in that the nucleic sequence of the gene coding for said aminoacyl-tRNA synthetase includes at least one mutation in comparison with the corresponding wild-type gene, said mutation not having been introduced by a gene recombination technique.

Thus, the invention relates to a method of selecting cells based on the constitution by the cell of a metabolic pathway necessary for its growth, making it possible to obtain cells capable of producing a non-canonical acyl-tRNA capable of charging a non-conventional amino acid.

Of the cells according to the invention, bacterial cells are preferred, characterised in that they are chosen from the following cells deposited in the CNCM (Collection Nationale de Culture de Microoganismes, Paris, France):

a) E coli strain deposited in the CNCM under no. I-2467 on Apr. 28, 2000,

b) E. coli strain deposited in the CNCM under no. I-2468 on Apr. 28, 2000,

c) E. coli strain deposited in the CNCM under no. I-2469 on Apr. 28, 2000, and

d) E. coli strain deposited in the CNCM under no. I-2470 on Apr. 28, 2000,

The E. coli strain K12, deposited in the CNCM under no. I-2467 and identified under reference β5419, the initial strain for making the selections, is a descendant of the strain MG1655 (wt E. coli K12), comprising the following characteristics:

deletion at the locus thyA and replacement by an erythromycin-resistant gene,

deletion at the locus nrdD and replacement by a kanamycin-resistant gene,

carries a pTZ18 plasmid (col E1 replicon, bla⁺) with the allele Cys146GUA of thymidylate synthase.

The strain E. coli K12, deposited in the CNCM under no. I-2468 and identified under the reference β5456, is a descendant of the strain MG1655 (wt E. coli K12), comprising the following characteristics:

deletion at the thyA locus and replacement by an erythromycin-resistant gene,

deletion at the nrdD locus and replacement by an kanamycin-resistant gene,

carries a pTZ18 plasmid (col E1 replicon, bla⁺) with the allele Cys146GUA of thymidylate synthase,

carries the allele T222P of the valS gene, the expression of which produces a mutated form of the valyl-tRNA synthase which charges tRNA/Val with other natural and artificial amino acids.

The strain E. coli K12, deposited in the CNCM under no. I-2470 and identified under the reference β5520, is a descendant of the strain MG1655 (wt E. coli K12), comprising the following characteristics:

deletion at the locus thyA and replacement by an erythromycin-resistant gene,

integration of a tetracyclin-resistant gene at the locus cycA30::Tn10,

carries a pTZ18 plasmid (col E1 replicon, bla⁺) with the allele Cys146GUA of thymidylate synthase,

carries the allele K277Q of the valS gene, the expression of which produces a mutated form of the valyl-tRNA synthase which charges tRNA/Val with other natural and artificial amino acids.

The strain E. coli K12, deposited in the CNCM under no. I-2469 and identified under the reference β5498, is a descendant of the strain CU505, comprising the following characteristics:

deletion at the locus nrdD and replacement by a kanamycin-resistant gene,

carries the allele T222P of the valS gene,

strain of the genotype leu-455 galT12 LAM-IN (rrnD-rrnE)1 DE (ilvE-ilvC) nrdD::kan valS:T222P,

strain deficient in the biosynthesis of valine and proficient in the misincorporation of L-alpha amino butyric acid in proteins by valine substitution.

The invention further includes the use of a method or of a cell according to the invention for the production of protein, in particular recombinant protein, whereof the sequence of amino acids includes at least one non-conventional amino acid.

From another perspective, the invention relates to a method for production of a protein whereof the sequence of amino acids includes at least one non-conventional amino acid characterised in that it includes the following steps:

a) where appropriate, selection of a cell by a method according to the invention;

b) culture of said cell selected at stage a) or of a cell according to the invention in a culture medium and culture conditions allowing the growth of said cell; and

c) isolation of said protein comprising at least one non-conventional amino acid from the culture supernatant and/or the cellular residue obtained at stage b).

In a preferred embodiment, the invention relates to a method for producing a protein whereof the sequence of amino acids includes at least one non-conventional amino acid characterised in that it includes the following steps:

a) the culture of a cell chosen from the following cells deposited in the CNCM (Collection Nationale de Culture de Microorganismes, Paris, France):

E. coli strain deposited in the CNCM under no. I-2467 on Apr. 28, 2000;

E. coli strain deposited in the CNCM under no. I-2468 on Apr. 28, 2000;

E. coli strain deposited in the CNCM under no. I-2469 on Apr. 28, 2000,

E. coli strain deposited in the CNCM under no. I-2470 on Apr. 28, 2000;

in a culture medium and culture conditions allowing the growth of said cell; and

b) isolation of said protein comprising at least one non-conventional amino acid from the culture supernatant and/or the cellular residue obtained at stage b).

Of the proteins which can be produced by a method according to the invention, mention may be made, but without being limited to these, of proteins which by the incorporation of at least one non-conventional amino acid make it possible to obtain a desired activity which a protein whereof the sequence consists solely of conventional amino acids does not make it possible to obtain. The term activity is intended to refer, in a general manner, to any activity such as a physiological or biological activity relative to uni- or multicellular organisms, even partial, such as for example a structural or biochemical activity, e.g. enzymatic, antigenic, of the antibody type, or modulation, regulation or inhibition of biological activity, or else such that it allows its utilisation in a biosynthesis or biodegradation process of chemical or biochemical compounds.

Of the proteins which can be produced by a method according to the invention, mention can also be made of proteins wherein the incorporation of at least one non-conventional amino acid is carried out in such a manner that it does not result in any essential modification of the biological activity of the corresponding unmodified protein. Besides the biological activity retained by the corresponding unmodified protein, these proteins according to the invention will present a non-conventional amino acid whereof the specific properties can be advantageously exploited.

Of the specific properties conferred by the presence of a non-conventional amino acid, mention may be made in particular of the properties linked to the presence of a functional group on said non-conventional amino acid capable of reacting easily and in a specific manner with a chemical or biochemical compound in conditions not allowing alteration of the activity of the protein or avoiding modification of the conventional amino acids.

The presence of this specific functional group can be used advantageously, for example to:

(i) purify any protein, in particular any recombinant protein, incorporating said non-conventional amino acid;

(ii) bind such a protein to a solid support;

(iii) bind to such a protein molecules capable of being detected, such as spectroscopic probes of various kinds;

(iv) bind to such a protein lipophilic or hydrophilic polymers enabling them to be solubilised in solvents or shielding them from recognition by antibodies;

(ii) bind such a protein to a polynucleotide;

(vi) bind such a protein to a chemical or biochemical compound whose presence makes it possible to increase, reduce, modulate, regulate or target the biological activity of said protein, or to modify its bioavailability as a compound with therapeutic use; or else

(vii) to fix in a permanent manner to such a protein a coenzyme which would otherwise diffuse in solution.

According to the present invention, the incorporation of at least one non-conventional amino acid can concern amino acids at the origin of a specificity or activity, or at the origin of the structural conformation, charge, hydrophobicity, or multimerisation capacity of the corresponding non-modified protein. It will thus be possible to create proteins of equivalent, increased or reduced activity, or of equivalent, narrower or wider specificity relative to the corresponding unmodified protein with conventional amino acids.

The term unmodified protein is intended to refer to the wild-type or recombinant protein made up of conventional amino acids, from which the protein comprising the non-conventional amino acid is produced.

The method of production according to the invention is preferably characterised in that said culture medium of stage b) allowing the growth of said cell contains said non-conventional amino acid or one of its precursors.

According to a particular embodiment, a method of production according to the invention is characterised in that said non-conventional amino acid is synthesised by said cell, it being possible to increase the synthesis of said non-conventional amino acid by genetic modification of said cell.

The invention further relates to a method of producing a protein whereof the amino acid sequence comprises at least one non-conventional amino acid according to the invention, characterised in that said cell is auxotrophic for the amino acid coded by said target codon.

Also included in the present invention are methods according to the invention, characterised in that said cell includes a gene of homologous or heterologous interest, whereof the coding sequence includes at least one target codon.

Generally speaking, the gene of interest will code for a messenger RNA which will then be translated into a protein of interest.

The gene of interest can be isolated by any conventional technique, such as cloning, PCR (Polymerase Chain Reaction) or else chemically synthesised. It may be of genomic type (having one or more introns) or complementary DNA (cDNA). The protein of interest can be constituted by a mature protein, a precursor, and in particular a precursor designed to be secreted and comprising a signal peptide, a truncated protein, a chimeric protein produced by the fusion of sequences of different origins or else a mutated protein having improved and/or modified biological properties.

Generally speaking, the gene of homologous or heterologous interest can be chosen from the genes coding for any protein which can be used as a therapeutic or cosmetic compound, or as a diagnostic reagent, or else as a compound which can be utilised in a biosynthesis or biodegradation process.

As examples, mention may be made of the genes of interest coding for the following proteins of interest:

cytokines or lymphokines (interferons α, β and γ, interleukines and in particular IL-2, IL-6, IL-10 or IL-12, tumour necrosis factors (TNF), colony stimulating factors (GM-CSF, C-CSF, M-CSF, etc.);

cellular or nuclear receptors, in particular those recognised by pathogenic organisms (viruses, bacteria or parasites) or ligands thereof;

proteins involved in a genetic disease (factor VII, factor VIII, factor IX, dystrophin or minidystrophin, insulin, CFTR (Cystic Fibrosis Transmembrane Conductance Regulator) protein, growth hormones (hGH);

enzymes (urease, renin, thrombin etc.) or any enzymes involved in the metabolism or biosynthesis of proteins, lipids, nucleic acids, sugars, amino acids, fatty acids or nucleotides;

enzyme inhibitors (α1-antitrypsin, antithrombin III, viral protease inhibitors etc.);

anti-tumour compounds capable of at least partially inhibiting the initiation or progression of tumours or cancers (antibodies, inhibitors acting at the level of cell division or transduction signals, expression products of tumour-suppressing genes, for example p53 or Rb, proteins stimulating the immune system etc.);

class I or II major histocompatibility complex proteins, or regulating proteins acting on the expression of the corresponding genes;

proteins capable of inhibiting a viral, bacterial or parasitic infection or its development (antigenic proteins having immunogenic properties, antigenic epitopes, antibodies etc.);

toxins such as ricin, cholera toxin, diphtheria toxin etc., or immunotoxins;

markers (β-galactosidase, peroxidase etc.); and

luciferase, GFP (green fluorescent protein), etc.

The invention also includes a method for producing a protein according to the invention, characterised in that the culture medium of stage b) additionally contains compounds necessary for induction of the synthesis of the protein coded by said gene of interest. These compounds are known to the person skilled in the art and depend in particular on the cell and homologous or heterologous gene selected.

The invention also concerns a method according to the invention, characterised in that the biological activity of the protein coded by said gene of interest is at least partially retained after incorporation of said non-conventional amino acid at the target codon of said gene of interest.

The invention also concerns a method according to the invention, characterised in that the non-conventional amino acid is chosen from the non-conventional amino acids of formula I and configuration L

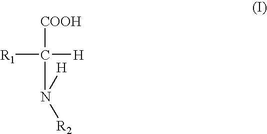

in which:

R₁ or R₂ represents radicals containing a functional group capable of reacting in a selective manner, preferably chosen from the aldehyde, ketone, ethenyl, ethynyl or nitrile groups.

Out of these groups, the oxo group (aldehyde or ketone) is particularly preferred, having selective reactivity which would facilitate the chemical functionalisation of the proteins. Other simple groups such as the ethynyl group would also lend themselves to selective reactions. A vast number of experiments carried out using acellular translation systems (ex vivo) and acyl-tRNA's synthesised in vitro, have shown that a great variety of acyl groups could be transferred to the ribosome in response to a codon read by the tRNA. In brief, lateral modifications to the amino acids all seem to be compatible with translation (to date no amino acid has been found whose lateral chain would be bulky enough to block translation); substitutions of the amino motif for alkyl-amino, hydroxy and hydrazino are compatible with the chemistry of transpeptidation catalysed by the ribosome (Bain et al. 1991) (it is known that the ribosome can form polyesters as well as conventional polyamides); replacement of the alpha hydrogen of the motif H₂NCH(R)—COOH by an alkyl (methyl) group or inversion of configuration at the alpha carbon (D-amino-acids) are not however accepted by the ribosome.

The invention also concerns a method according to the invention, for the functionalisation of protein.

The invention also concerns a method of purifying protein, characterised in that it comprises the following steps:

a) incorporation in the amino acid sequence of said protein, by a method according to the invention, of a non-conventional amino acid containing a functional group capable of reacting in a selective manner;

b) bringing the solution containing the protein obtained at stage a) into contact with a support comprising a compound capable of reacting specifically with said functional group and specifically fixing said protein; and

c) isolation of said protein fixed on the support.

The methods of purifying natural or recombinant protein normally used by the person skilled in the art generally involve methods used individually or in combination such as fractionation, chromatographic methods, immuno-affinity techniques using specific mono- or polyclonal antibodies etc. These methods are sometimes lengthy and tedious and do not always make it possible to obtain the specific activity, or the rate and yield of purification desired. The presence of a specific functional group on the protein to be purified, capable of reacting selectively with the purification support without altering the activity of the protein would considerably facilitate the purification of protein necessary for their use.

The invention also concerns a method of fixing a protein on a chemical or biochemical compound, characterised in that it comprises the following steps:

a) incorporation in the amino acid sequence of said protein by a method according to the invention of a non-conventional amino acid containing a functional group capable of reacting in a selective manner;

b) bringing the protein obtained at stage a) into contact with said chemical or biochemical compound comprising a group capable of reacting specifically with said functional group in a medium allowing the reaction.

The fixation of a protein on a chemical or biochemical compound is preferably a covalent bond fixation.

The chemical or biochemical compounds which can be used in said method of fixation according to the invention could be chosen from all the compounds capable of reacting with the functional group of the non-conventional amino acid incorporated.

In the present description, the term proteic complex is intended to refer to the product obtained at stage b) of the method described above, comprising a protein according to the invention fixed on a chemical or biochemical compound.

The invention also concerns a method according to the invention, characterised in that said chemical or biochemical compound is itself fixed on a solid support or is a compound constituting a solid support.

The invention also concerns a method according to the invention for preparing a proteic complex.

The invention preferably concerns the methods of the invention, characterised in that said fixed protein or said chemical or biochemical compound is chosen from therapeutic, cosmetic or diagnostic compounds.

Said fixed protein will in particular be chosen from the proteins whereof the amino acid sequence includes a non-conventional amino acid according to a method of the invention, and whereof the corresponding wild-type or recombinant non-modified protein is chosen from the proteins which can be used as therapeutic and cosmetic compounds or as diagnostic reagents.

The methods according to the invention are preferably characterised in that the chemical or biochemical compound is chosen from the compounds capable of modifying the biological activity of the fixed protein.

The term compounds capable of modifying the biological activity of another compound is intended to refer to a compound capable of increasing, reducing or regulating the biological activity of said other compound.

The invention also concerns a method according to the invention, characterised in that the chemical or biochemical compound is chosen from the compounds whose biological activity can be modified by the fixed protein.

The invention also concerns a method according to the invention, characterised in that the chemical or biochemical compound is chosen from the compounds including a protein, a polynucleotide, a fatty acid, a sugar or a natural or synthetic polymer.

From another perspective, the invention relates to proteins, in particular recombinant proteins, and proteic complexes obtained by a method according to the invention.

According to the present invention, the proteins obtained by a method of protein production of the invention will be recombinant in nature and their amino acid sequences will include at least one non-conventional amino acid.

According to the present invention, the proteic complexes obtained by a method for preparation of proteic complexes of the invention will in particular be characterised in that they include a recombinant protein whereof the amino acid sequence includes a non-conventional amino acid containing a functional group, and a chemical or biochemical compound comprising a group capable of reacting with said functional group.

The invention also concerns a method of selecting compounds capable of binding to a protein according to the invention or capable of binding to the chemical or biochemical compound of the proteic complex according to the invention. Of these methods, a method characterised in that it includes the following steps may be referred to as an example:

a) bringing said compound likely to be selected into contact with the protein or proteic complex according to the invention, said protein or proteic complex possibly being fixed in particular on a solid support;

b) determination of the capacity of said compound to bind with the protein or proteic complex according to the invention.

The compounds likely to be selected can be organic compounds such as proteins or carbohydrates or any other organic or inorganic compounds already known, or new organic compounds developed using molecular modelling techniques and obtained by chemical or biochemical synthesis, these techniques being known to the person skilled in the art.

The cells according to the invention can also advantageously serve as a model and be used in methods for studying, identifying and/or selecting proteins according to the invention or compounds likely to possess a desired activity.

The invention also relates to the use of a protein or a proteic complex according to the invention as a diagnostic reagent, as well as diagnostic methods, in particular for the detection, identification, localisation and/or specific dosage of polypeptide or polynucleotide, utilising a protein or a proteic complex according to the invention.

In effect, the proteins according to the invention include proteins having incorporated at least one non-conventional amino acid, and having partially or totally retained the initial activity of the corresponding unmodified wild-type or recombinant proteins, such as antibodies, antigens, enzymes or their biologically active fragments, known for being used in diagnostic methods.

In the same way, the proteic complexes according to the invention include proteic complexes formed from a protein according to the invention and a chemical or biochemical compound such as complexes comprising an antibody, antigen or oligonucleotide probe bound to an enzyme, a substrate or a molecule capable of being detected.

Of the diagnostic methods according to the invention, mention can be made for example of methods comprising the following steps:

a) bringing the biological sample likely to contain the desired compound into contact with a protein or a proteic complex according to the invention, said protein or proteic complex possibly being fixed in particular on a solid support; and

b) the detection, identification, location and/or dosage of the complex formed between the desired compound and a protein or a proteic complex according to the invention.

A person skilled in the art will be able to adapt the known standard diagnostic methods with the proteins or proteic complexes according to the invention.

The techniques and specific reagents allowing the detection, identification, location and/or dosage of the complex formed that can be used in the methods of the invention are also well known to a person skilled in the art, and are, for example ELISA, RIA, immunofluorescence, PCR techniques, or other techniques for amplification of a target nucleic acid known to a person skilled in the art.

The invention also relates to a diagnostic kit, in particular for the detection, identification, location and/or specific dosage of protein or polynucleotide characterised in that it contains a protein or proteic complex according to the invention.

The invention also relates to the use of a protein, a proteic complex or a cell according to the invention for the preparation of a pharmaceutical or cosmetic composition. The invention finally concerns a pharmaceutical or cosmetic composition comprising a protein, a proteic complex or a cell according to the invention.

Other characteristics and advantages of the invention are described below, with reference to the following examples:

### EXAMPLES

Characteristics of strains mentioned below in the examples.

The strain E. coli K12, deposited in the CNCM under no. I-2025 and identified under the reference β5366, is a descendant of the strain MG1655 (wt E. coli K12), comprising the following characteristics:

deletion at the locus thyA and replacement by an erythromycin-resistant gene,

carries a pTZ18 plasmid (col E1 replicon, bla⁺) with the allele Cys146GUA of thymidylate synthase.

The strain E. coli K12, deposited in the CNCM under no. I-2026 and identified under the reference β8144, is a descendant of the strain MG1655 (wt E. coli K12), comprising the following characteristics:

deletion at the locus thyA and replacement by an erythromycin-resistant gene,

carries a pTZ18 plasmid (col E1 replicon, bla⁺) with the allele Cys146GUA of thymidylate synthase.

The strain E. coli K12, deposited in the CNCM under no. I-2027 and identified under the reference β8146, is a descendant of the strain MG1655 (wt E. coli K12), comprising the following characteristics:

deletion at the locus thyA and replacement by an erythromycin-resistant gene,

carries a pTZ18 plasmid (col E1 replicon, bla⁺) with the allele Cys146GUA of thymidylate synthase.

The strain E. coli K12, deposited in the CNCM under no. I-2339 and identified under the reference β5479, is a descendant of the strain MG1655 (wt E. coli K12), comprising the following characteristics:

deletion at the locus thyA and replacement by an erythromycin-resistant gene,

deletion at the locus nrdD and replacement by a kanamycin-resistant gene,

carries the allele R223H of the valS gene,

carries a pTZ18 plasmid (col E1 replicon, bla⁺) with the allele Cys146GUA of thymidylate synthase.

The strain E. coli K12, deposited in the CNCM under no. I-2340 and identified under the reference β5485, is a descendant of the strain MG1655 (wt E. coli K12), comprising the following characteristics:

deletion at the locus thyA and replacement by an erythromycin-resistant gene,

deletion at the locus nrdD and replacement by a kanamycin-resistant gene,

carries the chromosome allele Val 276 Ala of the valS gene,

carries a pTZ18 plasmid (col E1 replicon, bla⁺) with the allele Cys146GUA of thymidylate synthase.

The strain E. coli K12, deposited in the CNCM under no. I-2341 and identified under the reference β5486, is a descendant of the strain MG1655 (wt E. coli K12), comprising the following characteristics:

deletion at the locus thyA and replacement by an erythromycin-resistant gene,

deletion at the locus nrdD and replacement by a kanamycin-resistant gene,

carries the chromosome allele Asp 230 Asn of the valS gene,

carries a pTZ18 plasmid (col E1 replicon, bla⁺) with the allele Cys146GUA of thymidylate synthase.

### EXAMPLE 1

### Construction of a Strain of E. Coli Comprising a False-Sense Mutation Cys->Val at the Active Site of the Thymidylate Synthase and Creating a Nutritional Requirement for Thymine, Thymidine or Cysteine

The artificial alleles of the thyA gene are constructed by directed mutagenesis of the pTSO plasmid (Lemeignan et al., 1993), which derives from the pTZ18R plasmid (BioRad) by insertion of the wild-type thyA gene of E. coli. The mutagenesis directed by means of an oligonucleotide is carried out according to the method described by Kunkel and coll. (1987) on the phagemid pTS0. Preparation of the single-strand matrix of pTS0, amplified in the strain RZ1032 (Kunkel and coll., 1987) (Hfr KL16 P045 [lysA(61-62)] dut1 ung1 thi1 relA1 supE44 zbd-279::Tn10) is carried out according to the procedure described by Sambrook and coll. (1989). A 5′ phosphorylated oligonucleotide (purchased from the company Genome Express) is used as mutagenic initiator:

Oligodeoxynucleotide 1 (SEQ ID NO: 1):

5′pTGGATAAAATGGCGCTGGCACCGGTACATGCATTCTTCCAGTTCTATGT.

The hybridation of this oligonucleotide with the single-strand matrix in each of the two constructions is carried out with 10 ng oligonucleotide and 0.2 μl matrix in a volume of 10 μl of a buffer solution containing 20 mM Tris-HCI pH 7.5, 2 mM EDTA and 50 mM sodium chloride. The tubes are incubated for 5 min at 70° C. then gradually cooled to 30° C. To this mixture is then added 0.5 mM of each of the dNTP's, 1 mM ATP, 10 mM Tris-HCI at pH 7.5, 10 mM magnesium chloride, 2 mM dithiothreitol and 1 unit of each of the two enzymes of the phage T4 DNA ligase and DNA polymerase. This reactive mixture of a final volume of 20 μl is incubated for 60 min at 37° C., of which 5 μl are then used to transform the competent cells of the strain GT869 (Parsot, C. 1986) (thrB1004 pro thi strA hsdS lacZ ΔM15 [F′ lacZ ΔM15 laclq traD36 proA+ proB+]) of E. coli K12 following the method described by Sambrook and coll. (1989). The cells transformed are spread out on Petri dishes containing the medium LB to which 100 mg/l of carbenicillin is added. Twelve clones resistant to the antibiotic are reisolated on the same medium. The single-strand DNA corresponding to the phagemids of these clones is prepared and sequenced according to the dideoxy method (Sanger and coll., 1977). The M13 sequencing kit, (Boehringer Mannheim, Mannheim, Germany) and the deoxyadenosine 5′-(α-thio)triphosphate (1300 Ci/mmol, Amersham) are combined according to the suppliers' instructions. Four initiators are used to determine the sequence of the thyA alleles:

Oligodeoxynucleotide 3 (SEQ ID NO: 3): 5′GGTGTGATCATGATGGTC

Oligodeoxynucleotide 4 (SEQ ID NO: 4): 5′CCTGCAAGATGGATTCCC

Oligodeoxynucleotide 5 (SEQ ID NO: 5): 5′CGCGCCGCATTATTGTTTC

Oligodeoxynucleotide 6 (SEQ ID NO: 6): 5′GTCTGGACCGGTGGCGACA

The plasmid pTS1 thus obtained propagates the allele thyA:Val146, in which the position 146 occupied in the wild-type thyA gene by the codon UGC of cysteine is occupied by the codon GUA of valine. The plasmid pTS1 is introduced by transformation, carried out according to the method of Sambrook and coll. (1989), in the strain ΔthyA of E. coli K12, β1308 (Lemeignan and coll., 1993), whereof the chromosome gene of the thymidylate synthase, thyA, is deleted. The transformed strain carrying the plasmidic allele thyA:Val146, β5366, proves to be incapable of growing without thymine or thymidine being added to the culture medium, as with the strain β1308 from which it derives. On the other hand, the strain β5366 shows marginal growth at 30° C. over a cysteine diffusion gradient, carried out in Petri dishes containing 25 ml of glucose mineral MS medium, from a central well containing 0.1 ml of a 400 mM solution of L-cysteine. Under the same conditions the strain β1308 does not give rise to any detectable growth. Thus the false-sense mutation converting the catalytic cysteine at position 146 of the thymidylate synthase into valine can be partially suppressed by a massive addition of exogenous cysteine. The addition of 0.1 mM valine to the Petri dish medium abolishes the growth of the strain β5366 over a cysteine gradient. Thus, everything happens as if the cysteine could infiltrate the active site of the valyl-tRNA synthetase to form erroneous Cys-tRNA^{val}'s capable of correcting the replacement of cysteine by valine in the active site of the thymidylate synthase. The excess valine would prevent the formation of these erroneous Cys-tRNA^{val}'s.

### EXAMPLE 2

### Construction of a Strain of E. coli Comprising a False-Sense Mutation Cys->Ile at the Active Site of the Thymidylate Synthase and Creating a Nutritional Requirement for Thymine, Thymidine, or Cysteine.

The corresponding construction is also carried out to replace the cysteine in position 146 with thymidylate synthase, by mutagenesis directed by means of the oligonucleotide 2, following the same procedure as in Example 1.

Oligodeoxynucleotide 2 (SEQ ID NO: 2):

5′pTGGATAAAATGGCGCTGGCACCGATACATGCATTCTTCCAGTTCTATGT

The plasmid pTS2 thus obtained propagates the allele thyA:Ile146, in which the position 146 occupied in the wild-type thyA gene by the codon UGC of cysteine is occupied by the codon AUA of isoleucine. The strain propagating the plasmidic allele thyA:Ile146, β35274, proves to require the nutritional addition of thymine, thymidine or cysteine in excess, as in the case of the strain β5366. Phenotypic suppression of the strain β5274 by cysteine is abolished by 0.1 mM isoleucine, just as that of the strain β5366 is abolished by valine. Thus everything happens as if the isoleucyl-tRNA synthetase was capable of forming erroneous Cys-tRNA^{Ile}'s in the presence of an excess of cysteine, and this erroneous formation was prevented by the presence of an excess of isoleucine.

### EXAMPLE 3

### Selection of Mutants of Genetic Code Misincorporating Cysteine Instead of Valine by Serial Culture in Liquid and Genetic Characterisation of the Mutants of the Valyl-tRNA Synthetase Thus Obtained.

The strain β5366 carrying the false-sense allele thyA:Val146 on the plasmid pTS1 is cultivated in a glucose mineral MS medium (2 g/l, Richaud and coll., 1993) supplemented with 0.3 mM thymidine for 24 hours at 30° C. in aerobiosis. The cells are then washed twice with deoxygenated mineral MS medium. A deoxygenated nutritive medium containing 10 ml glucose mineral MS medium to which 1.5 mM cysteine is added is inoculated at 1/100 using washed cells. The cells are then cultivated in anaerobiosis for 24 hours at 30° C. and a fresh tube containing 10 ml deoxygenated cysteine glucose mineral MS medium is inoculated with a 1/100 dilution of the culture at the preceding stationary phase. This procedure is repeated 26 times. At the end of this serial propagation, 12 clones from the liquid culture are isolated on dishes of thymidine (0.3 mM) glucose mineral MS medium (2 g/l) in aerobiosis and kept in suspension in the same liquid medium at −80° C. The twelve clones are tested on dishes containing glucose mineral MS medium with nutritional factors added. All these clones prove to require thymine or thymidine as a growth factor, unless cysteine is present in the culture medium, at a concentration of at least 1.5 mM.

Such clones are chosen for their thorough genetic characterisation, β8144 et β8146. Experiments with transduction by the phage P1 which is kanamycin-resistant in character, introduced into the locus nrdD, neighbour of the gene valS of valyl-tRNA synthetase (97 mn of the chromosome of E. coli K12) are carried out using strains β8144 and β8146. In both cases, approximately half the transducers resistant to kanamycin also exhibit nutritional dependence for thymidine suppressible by exogenous cysteine at a concentration of at least 1.5 mM. This proportion is in accordance with the genetic distance between the genes valS and nrdD (0.4 mn) and leads to the supposition that the phenotype of suppression of the false-sense mutation Cys->Val at the active site of the thymidylate synthase by low concentrations of cysteine is caused by genetic alteration of the locus valS. The fixation of a genetic alteration in the valS gene of the strains adapted is confirmed by sequencing of this locus: an A changed into a C causes the replacement of the lysine at position 277 by glutamine in the two adapted strains β8144 and β8146. The sequencing is carried out on a matrix obtained by polymerase chain reaction (PCR) carried out in the conditions described by Sambrook and coll. (1989). The amplification reaction is carried out in 100 μl of a solution containing 10 ng of genomic DNA of the strains β8144 or β8146, 20 pmoles of each initiator, 40 nmoles of an equimolar mixture of the 4 deoxynucleotide triphosphates, 10 μl of a buffer made up of 100 mM Tris-HCI pH 8.3, 500 mM KCI and 20 mM MgCl₂, in the presence of 1 to 2 units of Vent polymerase (Biolabs). For each reaction, 30 polymerisation cycles are completed, using a DNA amplifier (Perkin-Elmer Cetus), as follows: The denaturation is carried out at 94° C. for 5 min for the 1st cycle and 1 min for the following cycles, hybridisation at 58° C. for 1 min and elongation at 72° C. for 3 min for the first 29 cycles and for 10 min for the last cycle. The oligonucleotides 7 and 8 are used for amplification of the gene.

| | |
|---|---|
| Oligodeoxynucleotide 7: | |
| 5′GGGGAATTCGGTGTGTGAAATTGCCGCAGAACG | (SEQ ID NO:7) |
| Oligodeoxynucleotide 8: | |
| 5′GGCAAGCTTCCAGTATTTCACGGGGAGTTATGC | (SEQ ID NO:8) |

The PCR fragments thus obtained are purified using the QIAquick (Qiagen) kit and sent to the company Genaxis for determination of the sequence.

### EXAMPLE 4

### Phenotypic Suppression by Metabolic Precursors of Cysteine.

The nutritional requirement for cysteine of the adapted strains β8144 and β8146 is utilised to characterise metabolic precursors which can replace cysteine in the culture medium without giving rise to degradation by oxidation. S-carbamyl-L-cysteine (3 mM), S-methyl-L-cysteine (3 mM) and L-thiazolidine4-carboxylate acid (2 mM) have proved to be capable of replacing cysteine as a growth factor of the adapted strains β8144 and β8146, instead of thymidine or thymine. The same compounds prove to be capable of satisfying the cysteine requirement of a mutant cysN::kan (strain JT1, procured by M. Berlyn, Coli Genetic Stock Center, Yale University, USA (Levh et al., 1988)). However, the addition of none of these substances allows the growth of the strain β1308 leading to chromosomal deletion of the gene thyA of thymidylate synthase, thus excluding their contamination by traces of thymine or thymidine.

### EXAMPLE 5

### Selection of Mutants of the Genetic Code Misincorporating Cysteine Instead of Valine by Isolation on a Solid Medium and Genetic Characterisation of the Mutants of the Valyl-tRNA Synthetase Misincorporating Cysteine.

The strain β5366 carrying the false-sense allele thyA:Val146 on the plasmid pTS1 is transduced with a lysate of the phage P1 harvested on an auxiliary strain of E. coli (β7170, Bouzon et al., 1997) in the chromosome of which a marker of resistance to kanamycin had been introduced at the locus nrdD, neighbour of the locus valS of the gene of valyl-tRNA synthetase, thus producing the strain β5419. A mutator allele of the gene dnaQ is introduced extemporaneously by transduction of the strain β5419 using a lysate of the phage P1 harvested on an auxiliary strain (MS2131, Shapiro, 1990) carrying a marker of resistance to the tetracycline dnaQ::miniTn10. Such a clone which is resistant to tetracycline and exhibiting a rate of spontaneous mutation amplified approximately 1000 times (for acquisition of resistance to streptomycin) is cultivated at 30° C. in a minimum glucose medium in the presence of thymidine (0.3 mM). After 24 hours, the cells are harvested, and washed twice in an identical volume of culture medium without thymidine. A volume of 0.1 ml of the resultant suspension, corresponding to approximately 10⁸ bacteria, is spread on the surface of a series of Petri dishes containing a concentration of S-carbamyl-L-cysteine varying between 0 and 8 mM by adding 1 mM increments of the glucose mineral MS medium (2 g/l). The same procedure is applied to the non-mutator strain β5419, and to the wild-type gene dnaQ. All the Petri dishes are incubated for 96 hours at 30°. Colonies appear on the dishes having a concentration of S-carbamyl-L-cysteine exceeding 2 mM in the only case where the mutator allele dnaQ::miniTn10 was introduced into the strain tested.

A lysate of the phage P1 harvested from such a clone is used to transduce the strain β5366 carrying the plasmidic allele thyA:Val146. Approximately half the transducers resistant to kanamycin prove to be capable of growing in the presence of 3 mM S-carbamyl-L-cysteine and in the absence of thymine or thymidine, among them the strain β5455. The other half of the transducers are incapable of this, and require thymine or thymidine to proliferate, just like the strain β5366. This proportion of the phenotypes is in accordance with the genetic distance between the loci valS and nrdD (0.4 mn). Thus, suppression of the false-sense mutation of thyA Cys->Val by a low concentration of exogenous cysteine could result from an alteration of the gene of valyl-tRNA synthetase. The locus valS of one of the strains obtained by transduction of β5366 and capable of growing in the presence of 3 mM S-carbamyl-L-cysteine and in the absence of thymine or thymidine, designated β5455, is amplified by polymerase chain reaction and sequenced as described in Example 3. An A changed into a C causes replacement of the threonine at position 222 by proline, thus confirming the fixation of a genetic alteration in the gene valS of the strain β5455.

### EXAMPLE 6

### Sensitivity of the Mutants of Valyl-tRNA Synthetase to Non-Canonical Amino Acids.

The strains β5455, β8144 and β8146 are tested for their sensitivity to artificial amino acids which have a steric resemblance to valine. The test is carried out on dishes of glucose mineral MS medium supplemented with thymidine. The cells are cultivated in an aerobic medium (glucose mineral MS 0.3 mM thymidine) for 24 hours at 30° C. and diluted to 1/250 in the mineral MS medium. 0.5 ml of this cellular suspension is spread out on Petri dishes containing 25 ml g glucose mineral MS medium. A well is then hollowed out in the centre of the dish and filled with 0.1 ml of an amino acid solution:

(1) 100 mM L-2-amino-butyrate

(2) 100 mM L-2-amino-valerate

(3) 100 mM L-2-3-diamino-propionate

(4) 50 mM L-3-thiol-2-amino-butyrate.

The dishes are then incubated for 24 hours at 30° C. and the appearance, if any, of an inhibition zone on the dishes around the well is recorded. The diameters of the attenuated growth inhibition zones on the Petri dishes are measured:

L-2-amino-butyrate: 5.2 cm (β5455), 5.7 cm (β8144), 6.7 cm (β8146);

L-2-amino-valerate: 2.1 cm (β5455), 1.5 cm (β8144), 6.7 cm (β8146);

L-2-3-diamino-propionate: 2.3 cm (β5455), 2.7 cm (β8144), 1.9 cm (β8146);

L-3-thiol-2-amino-butyrate: 2.0 cm (β5366), 4.6 cm (β5455), 4.0 cm (β8144), 4.0 cm (β8146).

L-2-amino-butyrate, L-2-amino-valerate and L-2,3 diamino-propionate in the concentrations indicated are without effect on the strain β5366 at the wild-type valS allele, but inhibit the growth of the strains carrying a mutated valS gene. L-3-thiol-2-amino-butyrate inhibits the growth of all the strains, but the inhibition is more marked on the mutated strains. Thus everything happens as if the mutants of valyl-tRNA synthetase had an enlarged specificity making them capable of charging tRNA^{val}'s with amino acids which cannot be incorporated by the wild-type form of the enzyme.

### EXAMPLE 7

### Incorporation of the Non-Canonical Amino Acid α-aminobutyrate in the Proteins of an E. coli Strain Mutated in Valyl-tRNA Synthetase.

A lysate of phage P1 obtained from the strain β5455 (see example 5), was used to transduce the strain CU505 carrying an [symbol] ilvCABD deletion and a leu mutation making it auxotrophic for valine, isoleucine and leucine. The strain CU505 was obtained from the Coli Genetic Stock Center, at Yale University (USA). Transducing clones were selected from kanamycin LB dishes and tested for their sensitivity to amino-butyrate (3 mM) in a glucose MS solid medium (2 g/l) containing 0.3 mM of each of the three amino acids valine, isoleucine and leucine. Approximately 50% of the transducing clones could not grow in these conditions, indicating the co-transduction of the allele valS:T222P and the nrdD::kan resistance marker (see example 5). One of the transducing clones, designated β5498, was used to demonstrate the incorporation of amino-butyrate, replacing valine, in comparison with CU505. The two strains were cultivated at 30° C. in a glucose MS liquid medium (2 g/l) containing the dipeptide Ile-Leu at a concentration of 0.3 mM and the dipeptide Ile-Val at a concentration of 0.02 mM either in the presence of 0.2 mM L-amino-butyrate or in the absence of the analogue. The inoculum corresponding to each strain originated from a preculture in a glucose MS liquid medium (2 g/l) containing the dipeptide Ile-Leu at a concentration of 0.3 mM and the dipeptide Ile-Val at a concentration of 0.04 mM. The cultures (50 ml) in stationary phase after 24 hours at 30° C. were harvested by centrifugation. For each test, the residue was then resuspended in 25 ml of a trichloroacetic acid solution at 100 g/l (10% TCA) at 4° C., centrifuged, resuspended in 5 ml of 10% TCA, centrifuged once again, the residue resuspended in 5% TCA, the suspension incubated at 95° C. for 30 min, centrifuged, the residue resuspended in 5 ml acetone, centrifuged, the residue resuspended in 5 ml acetone, centrifuged, the residue resuspended in 5 ml acetone, centrifuged, and the residue left to dry. The residue thus obtained was dissolved in 1 ml of a solution of NH4HCO3 at 50 mM, to be lyophilised. The lyophilisate was dissolved in 2 ml 6N hydrochloric acid containing 2 g/l phenol, the mixture sealed in a phial, then incubated at 110° C. for 20 hours. The concentration of the amino acid in the hydrolysate was then quantified by derivatisation with ninhydrine following the instructions recommended by the supplier of the Beckman 6300 analyser. The amino-butyrate was detected in the hydrolysate of the proteins only in the case where the amino-butyrate had been added to the culture medium, and only for the strain β5498. The proportion of amino-butyrate replaced a quarter of the quantity of valine, corresponding to approximately 5 amino-butyrate residues per 100 amino acids of the total proteins. The detailed results of the analyses for the two strains CU505 and β5498 in the two culture conditions are given in the table below.

Chemical composition of the proteins extracted from strains auxotrophic for valine and cultivated with valine limitation, with or without amino-butyrate

| Amino acid | CU505 | β5498 | CU505 | β5498 |
|---|---|---|---|---|
| incorporated in | wt valS | valS T222P | wtvalS | valS T222P |
| the proteins | −Abu | −Abu | +Abu | +Abu |
| Abu | 0 | 0 | 0 | 0.20 |
| Val | 0.83 | 0.79 | 0.83 | 0.61 |
| Val + Abu | 0.83 | 0.79 | 0.83 | 0.81 |
| Ala | 1.32 | 1.28 | 1.32 | 1.22 |
| Ile | 0.61 | 0.61 | 0.61 | 0.61 |

Results expressed in Leu equivalents

### EXAMPLE 8

### Selection of New Mutants of the Genetic Code From a Mutator Strain by Isolation on a Solid Medium.

The strain β5419, expressing the inactive allele thyA:Val146 from a plasmid and carrying the marker [symbol] nrdD::kan in the chromosome, as accounted for by its construction described in Example 5, was transduced using a lysate of the phage P1 harvested on the strain TAD, carrying a mutator marker [symbol] mutS::spc, conferring resistance to spectinomycin, selecting on LB solid medium containing spectinomycin (25 mg/l) to obtain the strain β5555. The mutator phenotype of this strain was demonstrated by counting the frequency of mutants resistant to rifamycin. By following the experimental procedure described in Example 5, clones capable of growing at 30° C. in a glucose mineral medium without thymidine in the presence of 2 to 5 mM S-carbamoyl-L-cysteine (SCC), were obtained. Three of these clones served to prepare lysates of the phage P1, which were used to transduce the strain β5366, by selecting for resistance to kanamycin, following the procedure of Example 5. For each of the three lysates, approximately half the transducers were capable of growing in a glucose mineral solid medium containing 3 mM SCC, indicating the proximity of a mutation suppressing the false-sense allele thyA:C146V and of the marker nrdD::kan. The locus valS of the three strains β5479, β5485 and β5486, each corresponding to an SCC-suppressible transducer obtained from one of the three lysates was amplified by PCR and sequenced as described in Example 3. A different localised mutation was found for each of the three strains, viz. Arg 223 changed into His in the strain β5479, Val 276 changed into Ala in the strain β5485 and Asp 230 changed into Asn in the strain β5486. Thus, each clone having a phenotype of suppression of the false-sense mutant Cys 146 Val of thyA also shows sensitivity to amino-butyrate. Each of these clones has proved to carry a different localised mutation in the gene valS, validating the selective screen as a means of diversifying the activity of valyl-tRNA synthetase in Escherichia coli.

The mutant E. coli strains referenced β5456, β5520 and β5498, were obtained from the mutant strain β5419, as mentioned above in Example 5, and according to the selection procedures as described above in Examples 5 and 8.

### REFERENCES

BAIN J. D., E. S. DIALA, C. G. GLABE, D. A. WACKER, M. H. LYTTLE, T. A. DIX and A. R. CHAMBERLIN, 1991; Site-specific incorporation of nonstructural residues during in vitro protein biosynthesis with semisynthetic aminoacyl-tRNAs, Biochemistry 30:5411-5421.

BOUZON, M. and P. MARLIERE, 1997; Human deoxycytidine kinase as a conditional mutator in Escherichia coli. C.R. Acad.Sci. Paris 320:427434.

KUNKEL, T. A., and J. D. ROBERTS, 1987; Rapid and efficient site-specific mutagenesis without phenotypic selection. Methods Enzymol. 154:367-382.

LEMEIGNAN, B., P. SONIGO and P. MARLIERE, 1993; Phenotypic suppression by incorporation of an alien amino acid. J. Mol. Biol. 231:161-166.

LEVH, T. F., J. C. TAYLOR and G. D. MARKHAM, 1988; The sulfate activation locus of Escherichia coli K12: cloning, genetic, and enzymatic characterisation. J. Biol. Chem. 263:2409-2416.

PARSOT, C., 1986; Evolution of biosynthetic pathways: a common ancestor for threonine synthase, threonine dehydratase and D-serine dehydratase. EMBO J., 5:3013-3019.

RICHAUD, C., D. MENGIN-LECREULX, S. POCHET, E. J. JOHNSON, G. N. COHEN et al., 1993; Directed Evolution of Biosynthetic pathways. J. Biol. Chem. 268:26827-26835.

SAMBROOK, J., E. F. FRITSCH and T. MANIATIS, 1989; Molecular cloning: A Laboratory Manual. Cold Spring Harbor Laboratory Press, Cold Spring Harbor, NY.

SANGER, F., S. NICKLEN and A. R. COULSON, 1977; DNA sequencing with chain-terminating inhibitors. Proc. Natl. Acad. Sci. USA. 74:5463-5467.

SHAPIRO, J. A 1990; Action of a transposable element in coding sequence fusions. Genetics 126:293-299.

1. Method enabling cells to acquire the capacity to produce a protein whereof the amino acid sequence comprises at least one non-conventional amino acid, characterised in that it includes the following steps:
a) the transformation of said cells by at least one introduction of a false-sense mutation at a target codon of a gene coding for a protein necessary for the growth of said cells, said protein synthesised from the gene thus mutated no longer being functional;
b) where appropriate the culture of the cells obtained at stage a) in a culture medium containing a nutrient compensating for the loss of functionality of said protein thus mutated; and
c) culture of the cells obtained at stage a) or b) in a culture medium containing the amino acid coded by said target codon.

2. Method according to claim 1, characterised in that the culture medium of stage c) does not contain the nutrient necessitated by the loss of functionality of said mutated protein.

3. Method according to one of claims 1 and 2, characterised in that culture stage c) of said cells comprises a series of cultures of said cells in a culture medium containing the amino acid coded by said target codon, each of said cultures of the series being effected up to the obtaining of the stationary growth phase and followed by a washing of the cells obtained, the number of cultures of the series being sufficient to allow the selection of mutations increasing the suppression of said false-sense mutation of said mutated gene.

4. Method according to one of claims 1 to 3, characterised in that the false-sense mutation is chosen from the false-sense mutations which reverse spontaneously with only very low frequency, of the order of one organism out of at least 10¹⁵.

5. Method according to one of claims 1 to 4, characterised in that the false-sense mutation transforms a target codon of a gene coding for a protein necessary for the growth of said cell into a codon which in comparison with the target codon, presents a change of at least two bases, preferably three bases.

6. Method according to one of claims 1 to 5, characterised in that the target codon codes for an amino acid of low steric volume.

7. Method according to one of claims 1 to 6, characterised in that the target codon codes for an amphiphilic amino acid.

8. Method according to one of claims 1 to 7, characterised in that the target codon codes for an amino acid whose steric volume is lower than or roughly equal to the steric volume of the amino acid coded by the false-sense mutation.

9. Method according to one of claims 5 to 8, characterised in that the target codon codes for cysteine.

10. Method according to one of claims 5 to 9, characterised in that the amino acid coded by the false-sense mutation is valine or isoleucine.

11. Method according to one of claims 1 to 10, characterised in that the stage a) of the transformation of said cells is carried out by means of a vector comprising a sequence of said gene coding for a protein necessary for the growth of said cells comprising said false-sense mutation.

12. Method according to claim 11, characterised in that said vector is a plasmidic vector.

13. Method of selecting cells capable of producing a protein whereof the amino acid sequence includes at least one non-conventional amino acid characterised in that it comprises steps a), and where appropriate b) and c) of a method according to one of claims 1 to 12, and the selection of cells capable of growing at stage c).

14. Method of selecting cells according to claim 13, characterised in that it additionally includes a stage d) of culture of the cells obtained at stage c) in a culture medium containing said amino acid coded by said target codon, the concentration of said amino acid possibly being at a concentration higher than the concentration of said amino acid used in stage c), and the choice of cells sensitive to the concentration of said amino acids used in stage d).

15. Method of selecting cells according to one of claims 13 and 14, characterised in that the aminoacyl-tRNA synthetase recognising the amino acid coded by said false-sense mutation of said selected cells is capable of charging one of its associated tRNA's with a non-conventional amino acid or an amino acid other than said amino acid coded by said false-sense mutation.

16. Method of selecting cells according to claim 15, characterised in that the nucleic sequence of the gene coding for said aminoacyl-tRNA synthetase comprises at least one mutation in comparison with the corresponding wild-type gene sequence.

17. Method of selecting cells according to claim 16, characterised in that said mutation was not introduced by a gene recombination technique.

18. Cell obtained by a method according to one of claims 1 to 17.

19. Isolated cell capable of producing a protein whose amino acid sequence includes at least one non-conventional amino acid, characterised in that it includes an aminoacyl-tRNA synthetase recognising a given amino acid capable of charging one of its associated tRNA's with a non-conventional amino acid or an amino acid other than said given amino acid, and in that the nucleic sequence of the gene coding for said aminoacyl-tRNA synthetase includes at least one mutation in comparison with the corresponding wild-type gene sequence, said mutation not having been introduced by a gene recombination technique.

20. Cell according to claims 18 and 19, characterised in that it is chosen from the following cells deposited in the CNCM (Collection Nationale de Culture de Microorganismes, Paris, France):
a) E. coli strain deposited in the CNCM under no. I-2467 on Apr. 28, 2000,
b) E. coli strain deposited in the CNCM under no. I-2468 on Apr. 28, 2000,
c) E. coli strain deposited in the CNCM under no. I-2469 on Apr. 28, 2000, and
d) E. coli strain deposited in the CNCM under no. I-2470 on Apr. 28, 2000,

21. Use of a cell according to claim 20, for the production of protein whereof the amino acid sequence comprises at least one non-conventional amino acid.

22. Method for producing a protein whereof the amino acid sequence includes at least one non-conventional amino acid characterised in that it includes the following steps:
a) culture of a cell according to claim 20 in a culture medium and culture conditions allowing the growth of said cell; and
b) isolation of said protein comprising at least one non-conventional amino acid from the culture supernatant and/or the cellular residue obtained at stage b).

23. Method according to claim 22, characterised in that said culture medium of stage a) allowing the growth of said cell contains said non-conventional amino acid or one of its precursors.

24. Method according to claim 23, characterised in that said non-conventional amino acid is synthesised by said cell.

25. Method according to claim 24, characterised in that said non-conventional amino acid is augmented by genetic modification of said cell.

26. Method according to one of claims 22 to 25, characterised in that said cell is auxotrophic for the amino acid coded by said target codon.

27. Method according to one of claims 22 to 26, characterised in that said cell contains a gene of homologous or heterologous interest, whereof the coding sequence includes at least one target codon.

28. Method according to claim 27, characterised in that stage a) includes the compounds necessary for induction of the synthesis of the protein coded by said gene of interest.

29. Method according to claim 27 or 28, characterised in that the biological activity of the protein coded by said gene of interest is at least partially retained after incorporation of said non-conventional amino acid at the target codon of said gene of interest.

30. Method according to one of claims 22 to 29, characterised in that the non-conventional amino acid is chosen from the non-conventional amino acids of formula I of configuration L 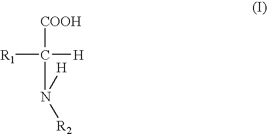
in which:
R₁ or R₂ represents radicals containing a functional group capable of reacting in a selective manner.

31. Method according to claim 30, characterised in that the functional group is chosen from the aldehyde, ketone, ethenyl, ethynyl or nitrile groups.

32. Method according to one of claims 23 to 31, for the functionalisation of protein.

33. Method for purifying protein, characterised in that it includes the following steps:
a) incorporation in the amino acid sequence of said protein of a non-conventional amino acid containing a functional group capable of reacting in a selective manner by a method according to one of claims 22 to 32;
b) bringing the solution containing the protein obtained at stage a) into contact with a support comprising a compound capable of reacting specifically with said functional group and specifically fixing said protein; and
c) isolation of said protein fixed on the support.

34. Method of fixing a protein on a chemical or biochemical compound, characterised in that it comprises the following steps:
a) incorporation in the amino acid sequence of said protein by a method according to one of claims 22 to 32 of a non-conventional amino acid containing a functional group capable of reacting in a selective manner;
b) bringing the protein obtained at stage a) into contact with said chemical or biochemical compound comprising a group capable of reacting specifically with said functional group in a medium allowing the reaction.

35. Method according to claim 34, characterised in that said chemical or biochemical compound is itself fixed on a solid support or is a compound constituting a solid support.

36. Method according to claim 34 or 35 for preparing a proteic complex.

37. Method according to one of claims 34 to 36, characterised in that the fixed protein or the chemical or biochemical compound is chosen from therapeutic, cosmetic or diagnostic compounds.

38. Method according to one of claims 34 to 37, characterised in that the chemical or biochemical compound is chosen from compounds capable of modifying the biological activity of the fixed protein.

39. Method according to one of claims 34 to 37, characterised in that the chemical or biochemical compound is chosen from compounds whose biological activity can be modified by the fixed protein.

40. Method according to one of claims 34 to 39, characterised in that the chemical or biochemical compound is chosen from the compounds including a protein, a polynucleotide, a fatty acid, a sugar or a natural or synthetic polymer.

41. Protein obtained by a method according to one of claims 22 to 33, characterised in that it concerns a recombinant protein whereof the amino acid sequence includes at least one non-conventional amino acid.

42. Proteic complex obtained by a method according to one of claims 34 to 40, characterised in that it includes a recombinant protein whereof the amino acid sequence includes a functional group and a chemical or biochemical compound comprising a group capable of reacting with said functional group.

43. Use of a protein according to claim 41, or of a proteic complex according to claim 42 as a diagnostic reagent.

44. Diagnostic method, characterised in that it utilises a protein according to claim 41, or a proteic complex according to claim 42.

45. Diagnostic kit, characterised in that it contains a protein according to claim 41, or a proteic complex according to claim 42.

46. Use of a protein according to claim 41, of a proteic complex according to claim 42 or a cell according to claim 20 for the preparation of a pharmaceutical or cosmetic composition.

47. Pharmaceutical or cosmetic composition comprising a protein according to claim 41, a proteic complex according to claim 42 or a cell according to claim 20.
